(19) United States
(12) Patent Application Publication
(10) Pub. No.: US 2025/0281572 A1
(43) Pub. Date: Sep. 11, 2025

(54) COMPOSITIONS AND METHODS FOR TREATING AND PREVENTING METABOLIC DISORDERS

(71) Applicant: The Johns Hopkins University, Baltimore, MD (US)

(72) Inventor: Ling He, Baltimore, MD (US)

(21) Appl. No.: 18/879,594

(22) PCT Filed: Jul. 28, 2023
(86) PCT No.: PCT/US2023/071191

§ 371 Date: Dec. 27, 2024

Related U.S. Application Data
(60) Provisional application No. 63/396,100, filed on Aug. 8, 2022.

(51) Int. Cl. A61K 38/17; A61K 38/10; A61P 35/00; C07K 7/08; C07K 14/47

(52) U.S. Cl. CPC A61K 38/1703; A61K 38/10; A61P 35/00; C07K 7/08; C07K 14/47

(57) ABSTRACT

Provided herein are compositions and methods for treating or preventing metabolic disorders and cancer. In particular, provided herein are compositions, methods, and uses of inhibitors of phosphorylation of AMPKalpha1 at Serine496 and AMPKalpha2 at S491 for improving metabolic function and treating and preventing metabolic diseases (e.g., diabetes and obesity) and cancer (e.g., hepatocellular carcinoma and breast cancer).

### STATEMENT OF RELATED APPLICATIONS

This application claims priority to U.S. Provisional Patent Application No. 63/396,100, filed Aug. 8, 2022, the entire contents of which are incorporated herein by reference for all purposes.

### STATEMENT OF GOVERNMENT SUPPORT

This invention was made with government support under grants nos. DK120309 and DK107641 awarded by the National Institutes of Health. The government has certain rights in the invention.

### FIELD

Provided herein are compositions and methods for treating or preventing metabolic disorders and cancer. In particular, provided herein are compositions, methods, and uses of inhibitors of phosphorylation of AMPKalpha1 at Serine496 and AMPKalpha2 at S491 for improving metabolic function and treating and preventing metabolic diseases (e.g., diabetes and obesity) and cancer (e.g., hepatocellular carcinoma and breast cancer).

### BACKGROUND

Diabetes mellitus type 2 is a long-term metabolic disorder that is characterized by high blood sugar, insulin resistance, and relative lack of insulin. Common symptoms include increased thirst, frequent urination, and unexplained weight loss. Symptoms may also include increased hunger, feeling tired, and sores that do not heal. Often symptoms come on slowly. Long-term complications from high blood sugar include heart disease, strokes, diabetic retinopathy which can result in blindness, kidney failure, and poor blood flow in the limbs which may lead to amputations. The sudden onset of hyperosmolar hyperglycemic state may occur; however, ketoacidosis is uncommon.

Type 2 diabetes is primarily due to obesity and not enough exercise in people who are genetically predisposed. It makes up about 90% of cases of diabetes, with the other 10% due primarily to diabetes mellitus type 1 and gestational diabetes. Diagnosis of diabetes is by blood tests such as fasting plasma glucose, oral glucose tolerance test, or HbA1c.

Type 2 diabetes is partly preventable by staying a normal weight, exercising regularly, and eating properly. Treatment involves exercise and dietary changes. If blood sugar levels are not adequately lowered, the medication metformin is typically recommended. Many people may eventually also require insulin injections. In those on insulin, routinely check blood sugar levels is advised, however this may not be needed in those taking pills. Bariatric surgery often improves diabetes in those who are obese.

Rates of type 2 diabetes have increased markedly since 1960 in parallel with obesity. As of 2013 there were approximately 368 million people diagnosed with the disease compared to around 30 million in 1985. Typically it begins in middle or older age. Type 2 diabetes is associated with a ten-year-shorter life expectancy.

New treatments for diabetes and associated conditions are needed.

### SUMMARY

Provided herein are compositions and methods for treating or preventing metabolic disorders and cancer. In particular, provided herein are compositions, methods, and uses of inhibitors of phosphorylation of AMPKalpha1 at Serine496 and AMPKalpha2 at S491 for improving metabolic function and treating and preventing metabolic diseases (e.g., diabetes and obesity) and cancer (e.g., hepatocellular carcinoma and breast cancer).

Impaired mitochondrial dynamics reduces nutrient metabolism and causes metabolic or aging related diseases. Yet, the molecular mechanism responsible for the impairment of mitochondrial dynamics is still not well understood. Experiments described herein demonstrated that that increased blood insulin and/or glucagon levels downregulate mitochondrial fission through directly phosphorylating AMPKα1S496 and/or AMPKα62S491 by either abnormally activated AKT or PKA, resulting in the impairment of AMPK-MFF-DRP1 signaling and mitochondrial oxidative activity. Peptides that block the phosphorylation of AMPKα1S496 and/or AMPKα2S491 were found to increase AMPK kinase activity and fragmentation of elongated mitochondria, improve mitochondrial oxidative activity, reduce ROS, and maintain healthy mitochondria population in hepatocytes prepared from obese mice and elderly mice or human subject. Furthermore, these peptides robustly suppressed glucose production in primary mouse and human hepatocytes and liver glucose production in obese mice. Further experiments demonstrated that the peptides inhibit the growth of tumor cells and fat accumulation.

Accordingly, in some embodiments, provided herein is a method of blocking the phosphorylation (e.g., inhibitory phosphorylation) of AMPKα1S496 and/or AMPKα2S491, comprising: contacting the AMPKα1/2 with a peptide that specifically (e.g., competitively and selectively) blocks phosphorylation of AMPKα1S496 and/or AMPKα2S491 at a substrate level (e.g., without affecting the upstream kinases' activity to avoid the unintended consequence when upstream AKT or PKA is inhibited). In some embodiments, the AMPKα1/2 is in a cell (e.g., in vivo or in vitro). In some embodiments, the contacting increases mitochondrial oxidative activity in the cell. In some embodiments, the contacting treats or prevents a metabolic disorder (e.g., including but not limited to, diabetes, obesity, cardiovascular diseases, or nonalcoholic fatty liver disease (NAFLD). In certain aspects, the contacting treats cancer.

Further provided herein is a method of improving one or more measures of mitochondrial function (e.g., including but not limited to, increasing mitochondrial respiration, enhancing mitochondrial fission, eliminating compromised mitochondria, improving mitochondrial oxidative state) in a subject, comprising: administering a peptide that specifically blocks phosphorylation of AMPKα1S496 and/or AMPKα2S491 to the subject.

Additionally provided is a method of treating or preventing a metabolic disorder in a subject, comprising: administering a peptide that specifically blocks phosphorylation of AMPKα1S496 and/or AMPKα2S491 to the subject.

Also provided is method of treating cancer in a subject, comprising: administering a peptide that specifically blocks phosphorylation of AMPKα1S496 and/or AMPKα2S491 to the subject.

In certain embodiments, the present disclosure provides the use of a peptide that specifically blocks phosphorylation of AMPKα1S496 and/or AMPKα2S491 to improve one or more measures of mitochondrial function, treat or prevent a metabolic disorder, or treat cancer in a subject.

The present disclosure is not limited to a particular peptide. Certain aspects of the technology utilize a peptide selected from, for example,

(SEQ ID NO: 1, termed Pa496h)
YGRKKRRQRRRTPQRSGSVSNYRSCQR;
(SEQ ID NO: 2, termed Pa496m)
YGRKKRRQRRRTPQRSGSISNYRSCQR;
(SEQ ID NO: 3, termed Pa2-491)
YGRKKRRQRRRTPQRSCSAAGLHR;
(SEQ ID NO: 4, termed a1h)
TPQRSGSVSNYRSCQR;
(SEQ ID NO: 5, termed a1m)
TPQRSGSISNYRSCQR;
(SEQ ID NO: 6 termed a2mh)
TPQRSCSAAGLHR,

or peptides with at least 90% (e.g., at least 90%, 91%, 92%, 93%, 94%, 95%, 96%, 97%, 98%, 99%, or 100%) identity to SEQ ID NOs: 1-6. In some embodiments, the peptide comprises, consisting essentially of, or consists of a peptide selected from SEQ ID NOs: 1-6.

In still other embodiments, provided herein is a composition, comprising a peptide selected

(SEQ ID NO: 1)
YGRKKRRQRRRTPQRSGSVSNYRSCQR;
(SEQ ID NO: 2)
YGRKKRRQRRRTPQRSGSISNYRSCQR;
(SEQ ID NO: 3)
YGRKKRRQRRRTPQRSCSAAGLHR;
(SEQ ID NO: 4)
TPQRSGSVSNYRSCQR;
(SEQ ID NO: 5)
TPQRSGSISNYRSCQR;
(SEQ ID NO: 6)
TPQRSCSAAGLHR

or peptides with at least 90% identity to SEQ ID NOs: 1-6. In some embodiments, the composition is a pharmaceutical composition.

Additional embodiments are described herein.

### DESCRIPTION OF THE FIGURES

FIG. 1 shows that mitochondria become elongated and exhibit reduced oxidative activity in elderly and obese mice.

FIG. 2 shows that insulin- and glucagon-mediated phosphorylation of AMPKα1S496 impairs AMPK-MFF signaling.

FIG. 3 shows that phosphorylation of AMPKα1S496 and/or AMPKα2S491 by either PKA or AKT impairs the phosphorylation of the protein at T172.

FIG. 4 shows that Pa496m and Pa496h augment mitochondrial respiration and triggers mitochondrial fission.

FIG. 5 shows that Pa496h treatment eliminates comprised mitochondria.

FIG. 6 shows that Pa496m and/or Pa496h suppress glucose production in hepatocytes and improved glucose tolerance, insulin sensitivity, and suppressed liver glucose production.

FIG. 7 shows that blocking AMPKα1/2 phosphorylation at S496/491 by Pa2-491 activates AMPK.

FIG. 8 shows that Pa2-491 triggers mitochondrial fission and suppresses glucose production in primary hepatocytes.

FIG. 9 shows that blocking AMPKα1/2 phosphorylation at S496/491 inhibits the growth of hepatoma HepG2 cells.

FIG. 10 shows that Pa2-491 stimulates the apoptosis in hepatoma HepG2 cells.

FIG. 11 shows suppression of breast cancer MCF-7 cell growth.

FIG. 12 shows that Pa496h stimulates the apoptosis in breast cancer MCF-7 cells.

FIG. 13 shows inhibition of colon Caco2 tumor cells' growth.

FIG. 14 shows that Pa496h stimulates the apoptosis in colon Caco2 tumor cells.

FIG. 15 shows inhibition of human kidney tumorigenic Hek293 cell growth.

FIG. 16 shows that Pa2-49 stimulates the apoptosis human kidney tumorigenic Hek293 cells.

FIG. 17 shows inhibition of human tumor cells growth using BrDu incorporation assay.

FIG. 18 shows that blocking AMPKol phosphorylation at S496 decreases lipid accumulation in hepatocytes.

FIG. 19 shows immunostaining with anti-AMPKα1S496 specific antibody in liver tissues from 4-month-old 4-month-old heterozygous lean and ob/ob mice (a), age-matched chow diet and HFD- (18 weeks) fed mice (b), and young and elderly mice (c).

FIG. 20 shows immunostaining with anti-AMPKαS496 specific antibody in the breast tissues from normal individual and patient with breast cancer, and liver tissues from normal individual and patient with liver cancer.

FIG. 21 shows Pa496h activates the signaling of apoptosis in breast cancer MCF-7 cells.

FIG. 22 shows overexpression of AMPK catalytic α1 or α2 subunit inhibits the growth of breast cancer cells and hepatoma cells.

FIG. 23 shows that AMPK-targeting peptides Pa496h and Pa2-491 inhibit the growth of human primary dissociated breast cancer dissociated tumor cells and hepatocellular carcinoma dissociated tumor cells.

FIG. 24 shows that lower concentrations of Pa496h and Pa2-491 inhibit the growth of human primary dissociated breast cancer dissociated tumor cells and hepatocellular carcinoma dissociated tumor cells.

FIG. 25 shows that AMPK-targeting peptides Pa496h and Pa2-491 inhibit the growth of breast cancer JIMT1 cells, colon carcinoma Caco2 cells, small cell lung cancer H69PR cells, B-lymphoma Ramos cells, melanoma HT144 cells, and pancreatic adenocarcinoma BxPC3 cells.

### DEFINITIONS

To facilitate an understanding of the present disclosure, a number of terms and phrases are defined below:

As used herein, the terms AMPKα1, AMPKα2, and AMPKα1/2 are used to refer to the alpha 1 and 2 subunits of protein kinase AMP-activated catalytic subunit alpha protein, encoded by the PRKAA1 and PRKAA2 genes (e.g., having accession numbers NM_006251.6 and NM_006252.4).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having a metabolic disease” refers to a subject that presents one or more symptoms indicative of a metabolic disease. A subject suspected of having a metabolic disease may also have one or more risk factors. A subject suspected of having metabolic disease has generally not been tested for metabolic disease. However, a “subject suspected of having metabolic disease” encompasses an individual who has received a preliminary diagnosis but for whom a confirmatory test has not been done or for whom the level or severity of metabolic disease is not known.

As used herein, the term “subject diagnosed with a metabolic disease” refers to a subject who has been tested and found to have a metabolic disease. As used herein, the term “initial diagnosis” refers to a test result of initial metabolic disease that reveals the presence or absence of disease.

As used herein, the term “subject at risk for metabolic disease” refers to a subject with one or more risk factors for developing a specific metabolic disease. Risk factors include, but are not limited to, gender, age, genetic predisposition, environmental exposure, and previous incidents of metabolic disease, preexisting non-fibrotic diseases, and lifestyle.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., fibrosis or cancer). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of a compound (e.g., a compound described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) (e.g., peptide described herein) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, or ex vivo.

As used herein, the term “toxic” refers to any detrimental or harmful effects on a cell or tissue as compared to the same cell or tissue prior to the administration of the toxicant.

A “peptide” or “polypeptide” is a linked sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide”, “oligopeptide,” and “peptide” are used interchangeably herein. The peptide(s) may be produced by recombinant genetic technology or chemical synthesis. The peptide(s) may be isolated and purified by any number of standard methods including, but not limited to, differential solubility (e.g., precipitation), centrifugation, chromatography (e.g., affinity, ion exchange, and size exclusion), or by any other standard techniques known in the art.

The recitations “sequence identity,” “percent identity,” “percent homology,” “percent similarity,” or, for example, comprising a “sequence 50% identical to” or “sequence with at least 50% similarity to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (e.g., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna. CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β3 and β2), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state. For example, the amino acid residue homoSerine (hSer) or homoSerine(Cl) in its unbonded form may take the form of 2-aminobutyric acid (Abu) when participating in an intramolecular bond according to the present invention.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or DF for the D isomeric form of Phenylalanine). Amino acid residues in the D isomeric form can be substituted for any L-amino acid residue, as long as the desired function is retained by the peptide.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), α-Glu (α-glutamic acid), Gaba (α-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,β diphenylalanine), and Ida (Iminodiacetic acid).

The term “pharmaceutically acceptable salt” in the context of the present invention (pharmaceutically acceptable salt of a peptide described herein) refers to a salt which is not harmful to a patient or subject to which the salt in question is administered. It may suitably be a salt chosen, e.g., among acid addition salts and basic salts. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the peptides may also be quaternized with alkyl chlorides, bromides, and iodides such as methyl, ethyl, propyl, isopropyl, butyl, lauryl, myristyl, stearyl and the like. Other examples of pharmaceutically acceptable salts are described in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977).

### DETAILED DESCRIPTION OF THE DISCLOSURE

Provided herein are compositions and methods for treating or preventing metabolic disorders and cancer. In particular, provided herein are compositions, methods, and uses of inhibitors of phosphorylation of AMPKalpha1 at Serine496 and AMPKalpha2 at S491 for improving metabolic function and treating and preventing metabolic diseases (e.g., diabetes and obesity) and cancer (e.g., hepatocellular carcinoma and breast cancer).

Mitochondria are the primary organelles responsible for nutrient metabolism, produce ATP, and also play a central role in controlling apoptosis, development, and reactive oxygen species levels, and integrating signaling pathways (Wallace, D. C. Mitochondrial diseases in man and mouse. Science 283, 1482-1488, doi:10.1126/science.283.5407.1482 (1999); Lopez, J. & Tait, S. W. Mitochondrial apoptosis: killing cancer using the enemy within. Br J Cancer 112, 957-962, doi:10.1038/bjc.2015.85 (2015); Munro, D. & Treberg, J. R. A radical shift in perspective: mitochondria as regulators of reactive oxygen species. J Exp Biol 220, 1170-1180, doi:10.1242/jeb.132142 (2017); Bohovych, I. & Khalimonchuk, O. Sending Out an SOS: Mitochondria as a Signaling Hub. Front Cell Dev Biol 4, 109, doi:10.3389/fcell.2016.00109 (2016); Hill, S. & Van Remmen, H. Mitochondrial stress signaling in longevity: a new role for mitochondrial function in aging. Redox Biol 2, 936-944, doi:10.1016/j.redox.2014.07.005 (2014)). Mitochondrial dysfunction causes serious health challenges and is a hallmark of metabolic diseases and aging (Abdul-Ghani, M. A. & DeFronzo, R. A. Mitochondrial dysfunction, insulin resistance, and type 2 diabetes mellitus. Curr Diab Rep 8, 173-178 (2008); Mansouri, A., Gattolliat, C. H. & Asselah, T. Mitochondrial Dysfunction and Signaling in Chronic Liver Diseases. Gastroenterology 155, 629-647, doi:10.1053/j.gastro.2018.06.083 (2018); Cheng, Z., Tseng, Y. & White, M. F. Insulin signaling meets mitochondria in metabolism. Trends Endocrinol Metab 21, 589-598, doi:10.1016/j.tem.2010.06.005 (2010); Petersen, K. F., Dufour, S., Befroy, D., Garcia, R. & Shulman, G. I. Impaired mitochondrial activity in the insulin-resistant offspring of patients with type 2 diabetes. N Engl J Med 350, 664-671, doi:10.1056/NEJMoa031314 (2004); Ritov, V. B. et al. Deficiency of subsarcolemmal mitochondria in obesity and type 2 diabetes. Diabetes 54, 8-14, doi:10.2337/diabetes.54.1.8 (2005); Sun, N., Youle, R. J. & Finkel, T. The Mitochondrial Basis of Aging. Mol Cell 61, 654-666, doi:10.1016/j.molcel.2016.01.028 (2016); Payne, B. A. & Chinnery, P. F. Mitochondrial dysfunction in aging: Much progress but many unresolved questions. Biochim Biophys Acta 1847, 1347-1353, doi:10.1016/j.bbabio.2015.05.022 (2015); Haas, R. H. Mitochondrial Dysfunction in Aging and Diseases of Aging. Biology (Basel) 8, doi:10.3390/biology8020048 (2019)). Mitochondria continually undertake fusion and fission processes to maintain a healthy mitochondrial population, and compromised mitochondria are eliminated via mitophagy (Youle, R. J. & van der Bliek, A. M. Mitochondrial fission, fusion, and stress. Science 337, 1062-1065, doi:10.1126/science.1219855 (2012); Liesa, M. & Shirihai, O. S. Mitochondrial dynamics in the regulation of nutrient utilization and energy expenditure. Cell Metab 17, 491-506, doi:10.1016/j.cmet.2013.03.002 (2013); Twig, G. et al. Fission and selective fusion govern mitochondrial segregation and elimination by autophagy. EMBO J 27, 433-446, doi:10.1038/sj.emboj.7601963 (2008); Lee, S. et al. Mitochondrial fission and fusion mediators, hFisl and OPA1, modulate cellular senescence. J Biol Chem 282, 22977-22983, doi:10.1074/jbc.M700679200 (2007); Vives-Bauza, C. et al. PINK1-dependent recruitment of Parkin to mitochondria in mitophagy. Proc Natl Acad Sci USA 107, 378-383, doi:10.1073/pnas.0911187107 (2010)). In mammals, mitochondrial fusion is mediated by mitofusins (MFN1/2) and Optic atrophy 1 (OPA1) (Eura, Y., Ishihara, N., Yokota, S. & Mihara, K. Two mitofusin proteins, mammalian homologues of FZO, with distinct functions are both required for mitochondrial fusion. J Biochem 134, 333-344, doi:10.1093/jb/mvg150 (2003); Santel, A. et al. Mitofusin-1 protein is a generally expressed mediator of mitochondrial fusion in mammalian cells. J Cell Sci 116, 2763-2774, doi:10.1242/jcs.00479 (2003); Alexander, C. et al. OPA1, encoding a dynamin-related GTPase, is mutated in autosomal dominant optic atrophy linked to chromosome 3q28. Nat Genet 26, 211-215, doi:10.1038/79944 (2000); Delettre, C. et al. Nuclear gene OPA1, encoding a mitochondrial dynamin-related protein, is mutated in dominant optic atrophy. Nat Genet 26, 207-210, doi:10.1038/79936 (2000); Malka, F. et al. Separate fusion of outer and inner mitochondrial membranes. EMBO Rep 6, 853-859, doi:10.1038/sj.embor.7400488 (2005)). Mitochondrial fission is mainly controlled by Mitochondrial fission factor (MFF) and Dynamin-related protein 1 (DRP1) (Twig et al., supra; Shin, H. W., Shinotsuka, C., Torii, S., Murakami, K. & Nakayama, K. Identification and subcellular localization of a novel mammalian dynamin-related protein homologous to yeast Vps1p and Dnm1p. J Biochem 122, 525-530, doi:10.1093/oxfordjournals.jbchem.a021784 (1997); Gandre-Babbe, S. & van der Bliek, A. M. The novel tail-anchored membrane protein MFF controls mitochondrial and peroxisomal fission in mammalian cells. Mol Biol Cell 19, 2402-2412, doi:10.1091/mbc.E07-12-1287 (2008)). DRP1 is mostly cytosolic, the phosphorylation of MFF at S155/172 by AMPK drives mitochondrial fission through recruiting DRP1 association with mitochondrial outer membrane-located MFF (Toyama, E. Q. et al. Metabolism. AMP-activated protein kinase mediates mitochondrial fission in response to energy stress. Science 351, 275-281, doi:10.1126/science.aab4138 (2016)). Mitochondrial biogenesis is also regulated by the PGCla-NRF1-TFAM signaling pathway (Puigserver, P. & Spiegelman, B. M. Peroxisome proliferator-activated receptor-gamma coactivator 1 alpha (PGC-1 alpha): transcriptional coactivator and metabolic regulator. Endocr Rev 24, 78-90, doi:10.1210/er.2002-0012 (2003); Luo, C., Widlund, H. R. & Puigserver, P. PGC-1 Coactivators: Shepherding the Mitochondrial Biogenesis of Tumors. Trends Cancer 2, 619-631, doi:10.1016/j.trecan.2016.09.006 (2016)).

Mitochondrial fusion can restore the functions of partially defective mitochondria by mixing contents (Youle et al., supra; Liesa et al., supra; Twig et al., supra; Lee et al., supra; Vives-Bauza et al., supra), promote increased efficiency of mitochondrial ATP synthesis to save nutrients, and is associated with cell senescence (Lesa et al., supra; Lee et al., supra; Yoon, Y. S. et al. Formation of elongated giant mitochondria in DFO-induced cellular senescence: involvement of enhanced fusion process through modulation of Fis1. J Cell Physiol 209, 468-480, doi:10.1002/jcp.20753 (2006)). In contrast, mitochondrial fission allows increased nutrient import and partial leak of the mitochondrial membrane potential, resulting in increased respiration along with a decrease in ATP synthesis efficiency (Molina, A. J. et al. Mitochondrial networking protects beta-cells from nutrient-induced apoptosis. Diabetes 58, 2303-2315, doi:10.2337/db07-1781 (2009)). Therefore, mitochondrial fission benefits cells by removing excess nutrients and their potentially cytotoxic metabolites and reducing ROS production in the presence of high caloric nutrients (Liesa et al., supra). Following the mitochondrial fission, compromised mitochondria are eliminated through mitophagy (Youle et al., supra; Liesa et al., supra; Twig et al., supra; Lee et al., supra; Vives-Bauza et al., supra). An abnormal mitochondrial life-cycle results in mitochondrial dysfunction, leading to a reduction in nutrient metabolism, accumulation of lipids, elevation of ROS levels in cells, and the development of metabolic or aging-related diseases (Hou, Y. et al. Ageing as a risk factor for neurodegenerative disease. Nat Rev Neurol, doi:10.1038/s41582-019-0244-7 (2019); Supinski, G. S., Schroder, E. A. & Callahan, L. A. Contemporary Review in Critical Care Medicine: Mitochondria and Critical Illness. Chest, doi:10.1016/j.chest.2019.08.2182 (2019); Akbari, M., Kirkwood, T. B. L. & Bohr, V. A. Mitochondria in the signaling pathways that control longevity and health span. Ageing Res Rev 54, 100940, doi:10.1016/j.arr.2019.100940 (2019); Sergi, D. et al. Mitochondrial (Dys)function and Insulin Resistance: From Pathophysiological Molecular Mechanisms to the Impact of Diet. Front Physiol 10, 532, doi:10.3389/fphys.2019.00532 (2019)).

AMPK is a principal energy sensing enzyme that is highly conserved and present in virtually all eukaryotes (Hardie, D. G., Ross, F. A. & Hawley, S. A. AMPK: a nutrient and energy sensor that maintains energy homeostasis. Nat Rev Mol Cell Biol 13, 251-262, doi:10.1038/nrm3311 (2012)). AMPK is consisted of two catalytic a subunits and two scaffold R subunits and three regulator γ subunits. Phosphorylation of a subunits at T172 by upstream kinases, such as the tumor-suppressor liver kinase B1 (LKB1), is critical for the activation of enzyme activity (subfamily, including MARK/PAR-1. EMBO J 23, 833-843, doi:10.1038/sj.emboj.7600110 (2004); Woods, A. et al. LKB1 is the upstream kinase in the AMP-activated protein kinase cascade. Curr Biol 13, 2004-2008, doi:10.1016/j.cub.2003.10.031 (2003)). Inappropriate hepatic glucose production is the major cause of hyperglycemia in patients with obesity and type 2 diabetes (T2D). Activation of AMPK can suppress liver gluconeogenesis (Takashima, M. et al. Role of KLF15 in regulation of hepatic gluconeogenesis and metformin action. Diabetes 59, 1608-1615, doi:10.2337/db09-1679 (2010); Wang, Y. et al. Metformin Improves Mitochondrial Respiratory Activity through Activation of AMPK. Cell Rep 29, 1511-1523 e1515, doi:10.1016/j.celrep.2019.09.070 (2019)) through increasing the phosphorylation of CBP at S436 and disassembly of CREB-coactivators complex (He, L. et al. Metformin and insulin suppress hepatic gluconeogenesis through phosphorylation of CREB binding protein. Cell 137, 635-646, doi:10.1016/j.cell.2009.03.016 (2009)). The phosphorylation of AMPKα1 at S496 (previously also designed as S485) by PKA reduces AMPKα1 at T172 and decreases AMPK enzyme activity (Cao, J. et al. Low concentrations of metformin suppress glucose production in hepatocytes through AMP-activated protein kinase (AMPK). J Biol Chem 289, 20435-20446, doi:10.1074/jbc.M114.567271 (2014); Djouder, N. et al. PKA phosphorylates and inactivates AMPKalpha to promote efficient lipolysis. EMBO J 29, 469-481, doi:10.1038/emboj.2009.339 (2010)). Activated AKT by insulin also phosphorylates AMPKα1 at Ser496 (Ning, J., Xi, G. & Clemmons, D. R. Suppression of AMPK activation via S485 phosphorylation by IGF-I during hyperglycemia is mediated by AKT activation in vascular smooth muscle cells. Endocrinology 152, 3143-3154, doi:10.1210/en.2011-0155 (2011); Hawley, S. A. et al. Phosphorylation by Akt within the ST loop of AMPK-alpha1 down-regulates its activation in tumour cells. Biochem J. 459(2):275-87. doi: 10.1042/BJ20131344 (2014)).

Experiments described herein demonstrated that that increased blood insulin and/or glucagon levels downregulate mitochondrial fission through directly phosphorylating AMPKα1S496 and/or AMPKα2S491 by activated AKT or PKA, resulting in the impairment of AMPK-MFF-DRP1 signaling and mitochondrial oxidative activity. Peptides that block AMPKα1S496 and/or AMPKα2S491 phosphorylation were found to increase AMPK kinase activity and fragmentation of elongated mitochondria, improve mitochondrial oxidative, reduce ROS, and maintain healthy mitochondria population in hepatocytes prepared from obese mice and elderly mice or primary human hepatocytes. Furthermore, these peptides robustly suppressed glucose production in hepatocytes prepared from obese mice, elderly mice, or primary human hepatocytes and liver glucose production in obese mice. Further experiments demonstrated that the peptides inhibit the growth of tumor cells and fat accumulation.

Accordingly, in some embodiments, provided herein is a method of blocking the phosphorylation of AMPKalpha1 at serine 496 and AMPKalpha2 at serine 491, comprising: contacting the AMPKalpha1 and 2 with a peptide that specifically blocks phosphorylation of AMPKalpha1 at serine 496 and AMPKalpha2 at serine 491. Example peptides and uses are described below.

### Peptides

The present disclosure is not limited to a particular peptide for blocking the phosphorylation of AMPKalpha1 at serine 496 and AMPKalpha2 at serine 491. Certain aspects of the technology utilize a peptide selected from, for example,

(SEQ ID NO: 1, termed Pa496h)
YGRKKRRQRRRTPQRSGSVSNYRSCQR;
(SEQ ID NO: 2, termed Pa496m)
YGRKKRRQRRRTPQRSGSISNYRSCQR;
(SEQ ID NO: 3, termed Pa2-491)
YGRKKRRQRRRTPQRSCSAAGLHR;
(SEQ ID NO: 4, termed a1h)
TPQRSGSVSNYRSCQR;
(SEQ ID NO: 5, termed a1m)
TPQRSGSISNYRSCQR;
(SEQ ID NO: 6 termed a2mh)
TPQRSCSAAGLHR,

or peptides with at least 90% (e.g., at least 90%, 91%, 92%, 93%, 94%, 95%, 96%, 97%, 98%, 99%, or 100%) identity to SEQ ID NOs: 1-6.

In some embodiments, peptides comprise a cell-penetrating sequence. Examples include but are not limited to, TAT (YGRKKRRQRRR; SEQ ID NO: 7), R8, MAP, Bip4, etc., (See e.g., Xie, J. et al. Cell-Penetrating Peptides in Diagnosis and Treatment of Human Diseases: From Preclinical Research to Clinical Application. Front Pharmacol 11, 697, doi:10.3389/fphar.2020.00697 (2020); herein incorporated by reference in its entirety).

In some embodiments, peptides comprise one or more substitutions relative to SEQ ID NOs: 1-6. In some embodiments, peptides comprise one or more (e.g., 1, 2, 3 4, 5, or more) insertions or additions to the C or N terminus of amino acids to the peptides of SEQ ID NOs: 1-6.

In some embodiments, the peptide comprises, consisting essentially of, or consists of a peptide selected from SEQ ID NOs: 1-6.

In some embodiments, peptides comprise one or more additional components useful in aiding the peptide in entering a cell. For example, in some embodiments, peptides are attached to a nanomaterial such as a nanoparticle (See e.g., ((Harish, V. et al. Review on Nanoparticles and Nanostructured Materials: Bioimaging, Biosensing, Drug Delivery, Tissue Engineering, Antimicrobial, and Agro-Food Applications. Nanomaterials (Basel) 12, doi:10.3390/nano12030457 (2022); herein incorporated by reference in its entirety).

Variants or analogs can differ from the peptides described herein by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Current Protocols in Molecular Biology” (Ausubel, 1987). Also included are cyclised peptides, molecules, and analogs which contain residues other than L-amino acids, e.g., D-amino acids or non-naturally occurring or synthetic amino acids, e.g., β or γ amino acids.

Amino acids include naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine. An amino acid analog is a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contains some alteration not found in a naturally occurring amino acid (e.g., a modified side chain); the term “amino acid mimetic” refers to chemical compounds that have a structure that is different from the general chemical structure of an amino acid, but that function in a manner similar to a naturally occurring amino acid. Amino acid analogs may have modified R groups (for example, norleucine) or modified peptide backbones, but retain the same basic chemical structure as a naturally occurring amino acid. In one embodiment, an amino acid analog is a D-amino acid, a β-amino acid, or an N-methyl amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Non-protein analogs having a chemical structure designed to mimic functional activity of the peptides described herein can be administered according to methods of the disclosure. Variants and analogs may exceed the physiological activity of the original peptide. Methods of analog design are well known in the art, and synthesis of analogs can be carried out according to such methods by modifying the chemical structures such that the resultant analogs exhibit the activity of a reference peptide. These chemical modifications include, but are not limited to, substituting alternative R groups and varying the degree of saturation at specific carbon atoms of a reference peptide. Preferably, the peptide analogs are relatively resistant to in vivo degradation, resulting in a more prolonged therapeutic effect upon administration. Assays for measuring functional activity include, but are not limited to, those described in the Examples below.

In some embodiments, candidate peptides are screened for activity (e.g., using the methods described the experimental section below or another suitable assay).

The present disclosure further provides pharmaceutical compositions (e.g., comprising the peptides described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present disclosure the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the peptide is administered in maintenance doses, ranging from 0.01 μg to 100 g per kg of body weight, once or more daily, to once every 20 years.

### Methods of Treating and Preventing Metabolic Disorders and Cancer

The peptides described herein find use in the treatment and prevention of metabolic disorders and cancers (e.g., including but not limited to, diabetes, obesity, cardiovascular diseases, or nonalcoholic fatty liver disease (NAFLD).

In some embodiments, the peptides improve one or more measures of mitochondrial function (e.g., including but not limited to, increasing mitochondrial respiration, enhancing mitochondrial fission, improving mitochondrial oxidative state) in a subject.

In some embodiments, the compounds and pharmaceutical compositions described herein are administered in combination with one or more additional agents, treatment, or interventions (e.g., agents, treatments, or interventions useful in the treatment of metabolic disorders). Examples include, but are not limited to, metformin, sulfonylureas, thiazolidinediones, dipeptidyl peptidase-4 inhibitors, SGLT2 inhibitors, glucagon-like peptide-1 analog, thiazolidinediones, angiotensin-converting enzyme inhibitors (ACEIs), insulin, and weight loss surgery.

### EXPERIMENTAL

The following examples are provided in order to demonstrate and further illustrate certain preferred embodiments and aspects of the present disclosure and are not to be construed as limiting the scope thereof.

### Example 1

### Methods

Adenoviruses and adeno-associated viruses (AAV). Adenovirus FLAG-tagged AMPKα1-WT and AMPKα1-S496A were generated as described previously (Cao, J. et al., Low concentrations of metformin suppress glucose production in hepatocytes through AMP-activated protein kinase (AMPK). J Biol Chem 289, 20435-20446, doi:10.1074/jbc.M114.567271 (2014)). The pAAV-TBG-EGFP expression vector was purchased from Vector Core at University of Pennsylvania. With permission, FLAG-tagged AMPKα1-WT and AMPKα1-S496A genes were used to replace EGFP and generated pAAV-TBG-AMPKα1-WT and pAAV-TBG-AMPKα1-S496A expression vectors.

Animal experiments. All animal protocols were approved by the Institutional Animal Care and Use Committee of the Johns Hopkins University. To generate the liver-specific AMPKα1/2 knockout mice with expression of AMPKα1-WT and -S496A mutant, double homozygous floxed AMPKα1/2 mice were injected with AAV8-TBG-Cre (2×10¹¹ GC/mouse) plus AAV8-TBG-FLAG-tagged AMPKα1-WT (3×10¹² GC/mouse), or AAV8-TBG-FLAG-tagged AMPKα1-S496A (3×10¹² GC/mouse) through the jugular vein.

Expression and purification of AMPK subunits and synthesis of peptides. The amounts of the adenoviral FLAG-tagged AMPKα1, AMPKβ1, and AMPKγ1 to achieve the expression rate of 1:1:1 were determined. These amounts of adenoviruses were added together for the purification of AMPKα1β1β1 heterotrimeric complex and the anti-FLAG antibody was used to immunoprecipitate the target proteins (Meng, S. et al., Metformin activates AMP-activated protein kinase by promoting formation of the alphabetagamma heterotrimeric complex. J Biol Chem 290, 3793-3802, doi: 10.1074/jbc.M114.604421 (2015)). The AMPK-targeting peptides were synthesized at the Sequencing and Synthesis Facility of Johns Hopkins School of Medicine.

Measurement of AMPK activity. For the measurement of cellular AMPK activity, cell lysates (500 ug) were incubated anti-AMPKα1 and α2 specific-antibodies (#3759, #3760, abcam) at 4° C. overnight, followed by the addition of protein G beads (Active Motif) to pull down the target protein and its associated proteins. The ADP-Glo kinase Assay kit was used to determine the utilization of ATP by the AMPK, followed the steps recommended by the manufacturer. Briefly, 5 ul of beads or purified AMPK proteins were incubated with (final concentration) 1× kinase buffer (Cell signaling), 0.32 mM SAMS peptide, 0.05 mM ATP at 37° C. for 60 min, then ADP-Glo was added and incubated at room temperature for 40 min. Kinase detection buffer was added and luminescence was determined 30 min later.

Determination of mitochondrial respiratory activity in primary hepatocytes. Primary hepatocytes were seeded in an XF 96 well plate coated with 0.01% collagen type I. 24 h after seeding, primary hepatocytes were treated as indicated, followed by the determination of mitochondrial respiratory chain activity using Seahorse XF96 Extracellular Flux Analyzers in Seahorse assay medium (12 mM pyruvate in base medium, pH7.4). After determination of basal oxygen consumption rates, cells were sequentially treated with oligomycin A (1 μM), FCCP (1 μM), and rotenone (1 μM) along with antimycin A (1 μM). Viable cell numbers were counted and used to normalize the oxygen consumption rate.

Glucose production assay and measurement of mitochondrial membrane potential and cellular ATP levels. Mouse primary hepatocytes were cultured in William's medium E supplemented with ITS (BD Biosciences) and dexamethasone (Cao, J. et al., Low concentrations of metformin suppress glucose production in hepatocytes through AMP-activated protein kinase (AMPK). J Biol Chem 289, 20435-20446, doi:10.1074/jbc.M114.567271 (2014)). After 24 h of seeding, cells were cultured in DMEM supplemented with 50 μM of TAT, Pa496m, or Pa496h for 16 h, and the medium was changed to FBS-free DMEM plus drugs. After 3 h of serum starvation, cells were washed twice with PBS, and cultured in the 1 mL glucose production medium (20 mM lactate, 2 mM lactate, pH7.4) plus drugs, and/or 10 nM glucagon or 0.2 mM Bt-cAMP. After 3 h incubation, both the medium and cells were collected. The medium was used to determine glucose concentrations with EnzyChrom Glucose Assay Kit. For the measurement of mitochondrial membrane potential, primary hepatocytes were stained with TMRE by using the Mitochondrial Membrane Potential Assay Kit (ab113852). The intensity of TMRE fluorescence was determined by microplate spectrophotometry (Wang, Y. et al., Metformin Improves Mitochondrial Respiratory Activity through Activation of AMPK. Cell Rep 29, 1511-1523, e1515, doi: 10.1016/j.celrep.2019.09.070 (2019)). Cellular ATP levels were determined using methods as reported previously (Cao, J. et al., Low concentrations of metformin suppress glucose production in hepatocytes through AMP-activated protein kinase (AMPK). J Biol Chem 289, 20435-20446, doi:10.1074/jbc.M114.567271 (2014)).

Confocal microscopy analysis. Twenty-four hours after the seeding, primary hepatocytes were cultured in DMEM or treated with indicated concentrations of TAT, Pa496m, and Pa496h for 16 h, and then the medium was changed with DMEM without phenol red supplemented with 50 nM MitoTracker™ Red FM (M22425, Thermo Fisher Scientific). Fluorescent images were acquired via a Zeiss confocal microscope (Zeiss Confocal LSM 880). The excitation wave lengths of MitoTracker™ Red CMR and Hoechst33342 are at 561 nm and 405 nm, respectively. Fifteen z-stacks were acquired, and then merged by Zeiss Zen software. If the length of the majority of the mitochondria was bigger than 10 μm, the cell was considered as cell with long mitochondria, otherwise, the cell was considered as a cell with short mitochondria (Wang, Y. et al., Metformin Improves Mitochondrial Respiratory Activity through Activation of AMPK. Cell Rep 29, 1511-1523, e1515, doi: 10.1016/j.celrep.2019.09.070 (2019)).

In vitro phosphorylation assays. Purified AMPKα1 protein was incubated with 1 μg of PKA catalytic subunit (Millipore) or 0.54 ug of AKT1 (abcam), 1× kinase buffer (Cell signaling) and 0.2 mM ATP at room temperature for the indicated time. To test the phosphorylation of PKA-mediated AMPK phosphorylation at S496/491 on a subunit phosphorylation at T172, AMPKα subunits were phosphorylated at S496/491 by PKA at 37° C. for 1 h, followed by the addition of 25 ng LKB1-STRAD-MO25 (Millipore), then incubated at room temperature for 30 min.

Immunohistochemistry. Frozen liver samples of db/db mice, ob/ob mice, aged mice, and HFD-fed mice were fixed with 4% paraformaldehyde in PBS for overnight at room temperature, then incubated in 30% sucrose overnight and frozen in OCT. These tissues were sectioned. After antigen retrieval using a microwave, sections were permeabilized with 0.1 Triton X-100 (10 min) and blocked in 3% horse serum for 1 h at room temperature. Paraffin-embedded human liver samples were sectioned. Sections were incubated with anti-AMPKα11S496-specific antibody (ab92701, abcam) at 4° C. overnight. For immunofluorescence staining, paraffin-embedded liver samples were sectioned, de-paraffinized, then antigen retrieval using high pressure steamer. Sections were blocked in 3% BSA and incubated with anti-PDH antibody (#110333, abcam), and then incubated with fluorescently-labeled secondary antibodies. Mitochondrial size was determined by using Image J ((Yamada, T. et al. Mitochondrial Stasis Reveals p62-Mediated Ubiquitination in Parkin-Independent Mitophagy and Mitigates Nonalcoholic Fatty Liver Disease. Cell Metab. 28(4):588-604 e5. doi: 10.1016/j.cmet.2018.06.014 (2018)).

ROS measurement and determination of mitophagic flux. After the treatment of primary hepatocytes, 5 μM CellRox Green (final concentration) (Molecular probes) was added to the cells and incubated at 37° C. for 30 min, washed with PBS, and the fluorescent intensity of ROS was measured by the Synergy H1 plate reader (BioTek). To determine the mitophagic flux, the mitochondrial-targeted Keima-Red gene (Safiulina, D. et al., Miro proteins prime mitochondria for Parkin translocation and mitophagy. EMBO J 38, doi: 10.15252/embj.201899384 (2019)) was cloned into a pENTR vector (Invitrogen) and transferred into the pAd/CMV vector (Invitrogen) by recombination to generate expression clones. The adenoviral Keima expression vector was added to the primary hepatocytes after 4 h seeding. 16 h after the addition of the viral vector, hepatocytes were treated with 100 μM of Pa496h. Keima-tagged mitochondria in cytoplasm or lysosome were determined by confocal microscope (Zeiss Confocal LSM 880) with excitation spectrum 480 and 561 nm respectively.

Statistical analyses. Statistical significance was calculated with a Student's t test and ANOVA test. Significance was accepted at the level of p<0.05. At least 3 samples per group were chosen for statistically meaningful interpretation of results and differences in the studies using the Student's t test and analysis of variation.

### Results

### Mitochondria Become Elongated and Exhibit Reduced Oxidative Activity in Elderly and Obese Mice

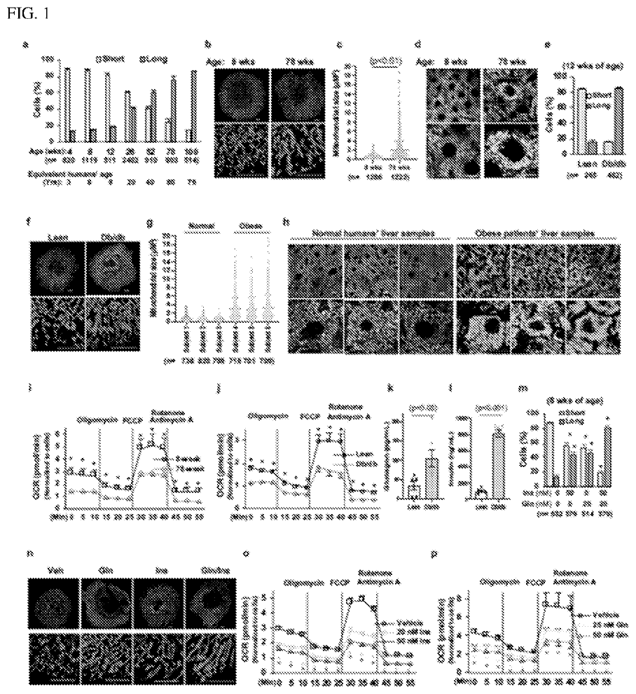

Patients with obesity and elderly individuals have compromised mitochondrial dynamics ((Morino, K. et al. Reduced mitochondrial density and increased IRS-1 serine phosphorylation in muscle of insulin-resistant offspring of type 2 diabetic parents. J Clin Invest 115, 3587-3593, doi:10.1172/JC125151 (2005); Jendrach, M. et al. Morpho-dynamic changes of mitochondria during ageing of human endothelial cells. Mech Ageing Dev 126, 813-821, doi:10.1016/j.mad.2005.03.002 (2005)). Mitochondrial morphology was examined in liver hepatocytes from mice with different ages (4 to 100 weeks of age), and it was found that in young mice (4-12 weeks of age), the majority (>80%) of the mitochondria is in a shorten form, however, mitochondria become elongated and form reticula with aging (FIGS. 1a, b). The mitochondrial sizes in the liver tissues of young (8-week of age) and elderly mice (78-week of age) were determined using immunofluorescence staining with specific antibodies against mitochondrial matrix protein pyruvate dehydrogenase (PDH) ((Li, B. et al. Mitochondrial-Derived Vesicles Protect Cardiomyocytes Against Hypoxic Damage. Front Cell Dev Biol 8, 214, doi: 10.3389/fcell.2020.00214 (2020); Yamada, T. et al. Mitochondrial Stasis Reveals p62-Mediated Ubiquitination in Parkin-Independent Mitophagy and Mitigates Nonalcoholic Fatty Liver Disease. Cell Metab 28, 588-604 e5, doi: 10.1016/j.cmet.2018.06.014 (2018)), it was found that the average mitochondrial size increased more than 3-fold in the liver of elderly mice than in the liver of young mice (FIGS. 1c, d). In the liver hepatocytes prepared from genetically obese db/db mice, the majority (>80%) of the mitochondria is in an elongated from and becomes reticula (FIGS. 1e, f). Consistently, the average mitochondrial size increased in the liver of obese patients (FIGS. 1g, h). These mitochondrial morphology changes are accompanied with decreased mitochondrial respiratory activity (oxygen consumption rate, OCR) in elderly mice and obese mice (FIGS. 1i, j). The above data indicate that mitochondria have lower oxidative activity when becoming elongated.

### Elevated Insulin and Glucagon Levels Promote the Elongation of Mitochondria

Blood insulin and/or glucagon levels elevate with age in mice and human subjects (Fan, R., Kang, Z., He, L., Chan, J. & Xu, G. Exendin-4 improves blood glucose control in both young and aging normal non-diabetic mice, possible contribution of beta cell independent effects. PLoS One 6, e20443, doi:10.1371/journal.pone.0020443 (2011); 48 Melanson, K. J., Saltzman, E., Vinken, A. G., Russell, R. & Roberts, S. B. The effects of age on postprandial thermogenesis at four graded energetic challenges: findings in young and older women. J Gerontol A Biol Sci Med Sci 53, B409-414, doi:10.1093/gerona/53a.6.b409 (1998); Stevic, R. et al. Oral glucose tolerance test in the assessment of glucose-tolerance in the elderly people. Age Ageing 36, 459-462, doi:10.1093/ageing/afm076 (2007); Chow, H. M. et al. Age-related hyperinsulinemia leads to insulin resistance in neurons and cell-cycle-induced senescence. Nature neuroscience 22, 1806-1819, doi:10.1038/s41593-019-0505-1 (2019)), and obese ob/ob mice (Dubuc, P. U. et al. Immunoreactive glucagon levels in obese-hyperglycemic (ob/ob) mice. Diabetes 26, 841-846, doi: 10.2337/diab.26.9.841 (1977)), and db/db have increased blood insulin and glucagon levels (FIGS. 1k, l). It was therefore tested whether higher concentrations of insulin and/or glucagon could affect mitochondrial dynamics, and it was found that indeed both insulin or glucagon along significantly increased the ratio of elongated mitochondria in primary hepatocytes prepared from young mice (2 months old) (FIGS. 1m, n). In addition, insulin and glucagon could synergistically increase the ratio of elongated mitochondria. Prolonged treatment with insulin (20 h) or glucagon (6 h) significantly reduced mitochondrial oxidative activity in a concentration-dependent manner (FIGS. 1o, p). These data indicate that elevated blood insulin and/or glucagon may be the “culprit” responsible for the formation of elongated mitochondria in aging and obesity.

### Elevated Insulin- and Glucagon-Mediated Phosphorylation of AMPKα1S496 Impairs AMPK-MFF Signaling

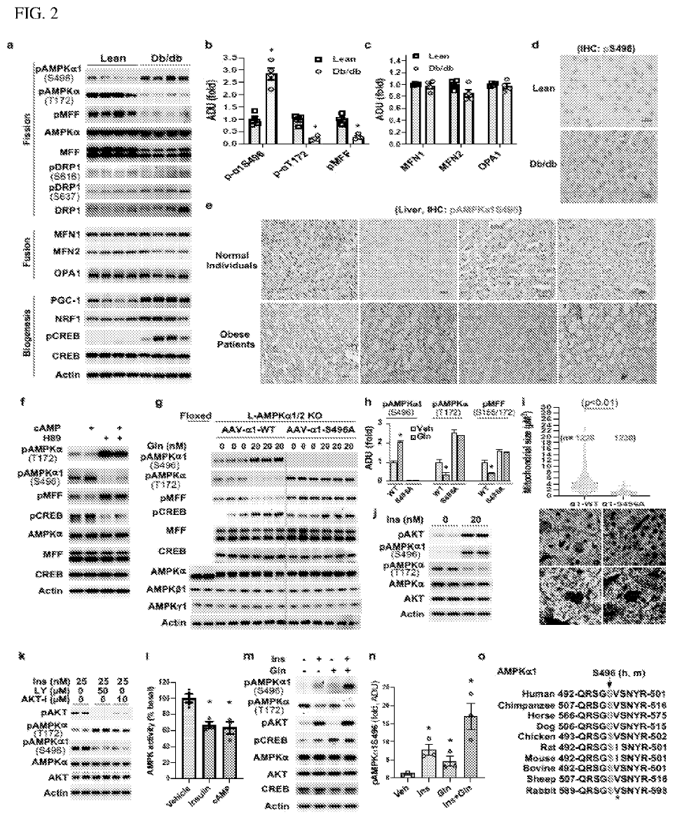

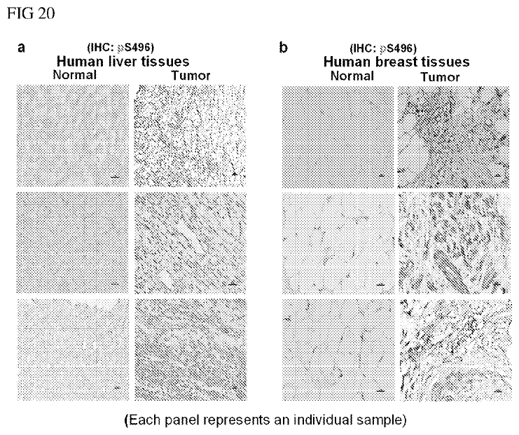

To identify the pathway leading to the formation of elongated mitochondria in liver hepatocytes of obese db/db mice (FIGS. 1e, f), the phosphorylation and protein levels of mediators that regulate mitochondria dynamics and population were examined. It was found that there were significant reductions of phosphorylation of AMPKαT172 and MFF in the liver of db/db mice compared to that of lean control mice (FIGS. 2a, b), showing an impaired mitochondrial fission because the activation of AMPK-MFF signaling plays a critical role in driving mitochondrial fission ((Toyama, E. Q. et al. Metabolism. AMP-activated protein kinase mediates mitochondrial fission in response to energy stress. Science 351, 275-281, doi:10.1126/science.aab4138 (2016)). In contrast, the protein levels of MFN1/2 and OPA1 had no significant changes in the liver of lean control mice and db/db mice (FIGS. 2a, c). An activation of PGCla-NRF1 signaling (FIG. 2a) was observed; this may be a compensatory response for the impairment of mitochondrial activity in this obese mouse model. However, AMPKα1S496 phosphorylation levels were significantly increased in the liver of db/db mice (FIGS. 2a, b). In addition, immunostained liver sections from db/db mice had increased AMPKα1 phosphorylation at S496 (FIG. 2d), and obese patients had significantly elevated liver AMPKα1 phosphorylation at S496 compared to normal individuals (FIG. 2e). Moreover, activation of cAMP-PKA signaling by cAMP (0.2 mM) increased AMPKα1S496 phosphorylation and decreased MFF phosphorylation in Hepa1-6 cells (FIG. 2f); in contrast, PKA inhibitor H89 reversed the negative effect of cAMP on the phosphorylation of AMPKαT172 and MFF. To validate the negative impact of S496 phosphorylation on AMPKα phosphorylation at T172, wild type AMPKα1 (dominant isoform of a subunit, accounting for over 90% of AMPK activity in the liver ((Davies, S. P. et al. Purification of the AMP-activated protein kinase on ATP-gamma-sepharose and analysis of its subunit structure. Eur J Biochem 223, 351-357, doi: (1994)) and mutant AMPKα1S496A to similar levels as its endogenous protein levels were expressed in the liver of liver-specific AMPKα1 and 2 knockout mice. Primary hepatocytes (FIG. 2g) were prepared from these mice. Hepatocytes with expression of mutant AMPKα1S496A had significantly higher phosphorylation levels of AMPKαT172 and MFF, and resisted glucagon-stimulated (20 nM) the phosphorylation of AMPKα1S496 and impairment of AMPKαT172 phosphorylation (FIGS. 2g, h). These data indicate that S496 phosphorylation in AMPKα1 exerts a negative effect on the phosphorylation at T172 in this protein. Furthermore, overexpression of AMPKα1S496A in the liver of obese db/db mice led to a significantly decreased size of liver mitochondria (FIG. 2i). Higher concentrations of insulin could stimulate the phosphorylation of AMPKα1S496 along with the reduction of AMPKαT172 phosphorylation, and both PI3K inhibitor LY294002 and AKT inhibitor AKT-i inhibited insulin signaling and AMPKα1S496 phosphorylation along with increased AMPKαT172 phosphorylation in primary hepatocytes (FIGS. 2j, k). Treatment with either 25 nM insulin or 0.4 mM cAMP significantly decreased AMPK enzymatic activity in Hepa1-6 cells (FIG. 2l). In addition, higher concentrations of insulin (25 nM) and glucagon (15 nM) have synergistic effect on stimulation of AMPKα1 phosphorylation at S496 in primary hepatocytes (FIGS. 2m, n). The phosphorylation site of AMPKα1 at S496 is conserved across species (FIG. 20).

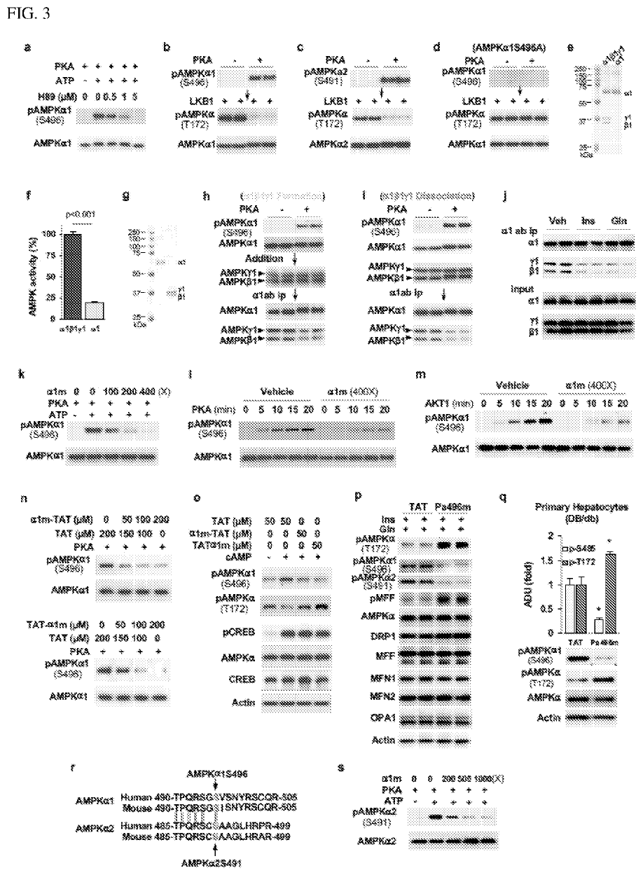

Phosphorylation of AMPKα1S496 Impairs the Phosphorylation of AMPKα at T172 PKA can directly phosphorylate AMPKα1 at S496 (FIGS. 3a, b) and AMPKα2 at S491 (FIG. 3c), and the phosphorylation at these sites decreased the phosphorylation of AMPKα at T172 by upstream kinase LKB1 (FIGS. 3b-d). The functional AMPK kinase is a heterotrimeric αβγ complex ((Hardie, D. G., Ross, F. A. & Hawley, S. A. AMPK: a nutrient and energy sensor that maintains energy homeostasis. Nat Rev Mol Cell Biol 13, 251-262, doi:10.1038/nrm3311(2012)), and AMPK lost near 80% of enzymatic activity in the absence of βγ subunit (FIGS. 3e, f). It was tested whether AMPKα1S496 phosphorylation had any effect on the formation of AMPK αβγ heterotrimeric complex, and it was found that AMPKα1S496 phosphorylation decreased the formation of α1β1γ1 heterotrimeric complex (FIGS. 3g, h). In addition, AMPKα1S496 phosphorylation facilitated the dissociation of α1β1γ1 heterotrimeric complex in in vitro assay (FIG. 3i). Treatment with either higher concentrations of insulin (25 nM) or glucagon (15 nM) reduced the binding of β1γ1 subunits to the α 1 subunit in Hepa1-6 cells (FIG. 3j).

### Generation of a Peptide to Block the Phosphorylation of AMPKα1 at S496 and AMPK α2 at S491

Having seen that the phosphorylation of AMPKα1 at S496 has a negative impact on AMPK activity (FIGS. 2g, h, 3b-d), a peptide a1m (TPQRSGSISNYRSCQR), which corresponds to mouse AMPKα1 from 490-505 a.a. to block the phosphorylation of AMPKα1 at S496, was generated. It was found that this peptide could effectively antagonize PKA- or AKT-mediated phosphorylation of AMPKα1 at S496 in in vitro assays (FIGS. 3k-m). To test whether this peptide could block AMPKα1S496 phosphorylation at cellular level, a cell-penetrating TAT sequence (YGRKKRRQRRR) ((Mishra, A. et al. Translocation of HIV TAT peptide and analogues induced by multiplexed membrane and cytoskeletal interactions. Proc Natl Acad Sci USA 108, 16883-16888, doi:10.1073/pnas.1108795108 (2011); Green, M. & Loewenstein, P. M. Autonomous functional domains of chemically synthesized human immunodeficiency virus tat trans-activator protein. Cell 55, 1179-1188, doi:10.1016/0092-8674(88)90262-0 (1988)) was added at either the N-terminal or N-terminal of a1m peptide. The addition of TAT sequence to the N- or C-terminal of a1m peptide did not affect the blockade of AMPKα1 phosphorylation of at S496 by a1m peptide in in vitro assays (FIG. 3n). However, the addition of TAT sequence at the N-terminal of a1m peptide had stronger inhibition of cAMP (0.2 mM) stimulated AMPKα1 phosphorylation of at S496 and increase in AMPKα phosphorylation at T172 in Hepa1-6 cells (FIG. 3o). This peptide was termed Pa496m (Peptide anti-phosphorylation of AMPKαS496, mouse). The Pa496m (50 μM) could effectively inhibited both insulin (25 nM) and glucagon (15 nM) stimulated AMPKα1S496 phosphorylation and increased the phosphorylation levels of AMPKα at T172 and MFF in primary hepatocytes (FIG. 3p). As shown in FIG. 3q, Pa496m (50 PM) also significantly decreased AMPKα1S496 phosphorylation along with increased AMPKα phosphorylation at T172 in primary hepatocytes prepared to db/db mice that have elevated AMPKα1S496 phosphorylation in the liver (FIGS. 2a, b).

Because the α1m (TPQRSGSISNYRSCQR) peptide could effectively antagonize PKA- or AKT-mediated phosphorylation of AMPKα1 at S496 in in vitro assays (FIGS. 3k-m), and due to the six identical amino acids at the N-terminus of inhibitory phosphorylation site of AMPKα1 at S496 and AMPKα2 at S491 were included in peptide α1m (FIG. 3r), this targeting-peptide a1m could antagonize the phosphorylation of AMPKα2 at S491 by PKA as well (FIG. 3s), and Pa496m was tested in studies of affecting mitochondrial activity.

### AMPK-Targeting Peptides Augment Mitochondrial Respiration and Triggers Mitochondrial Fission

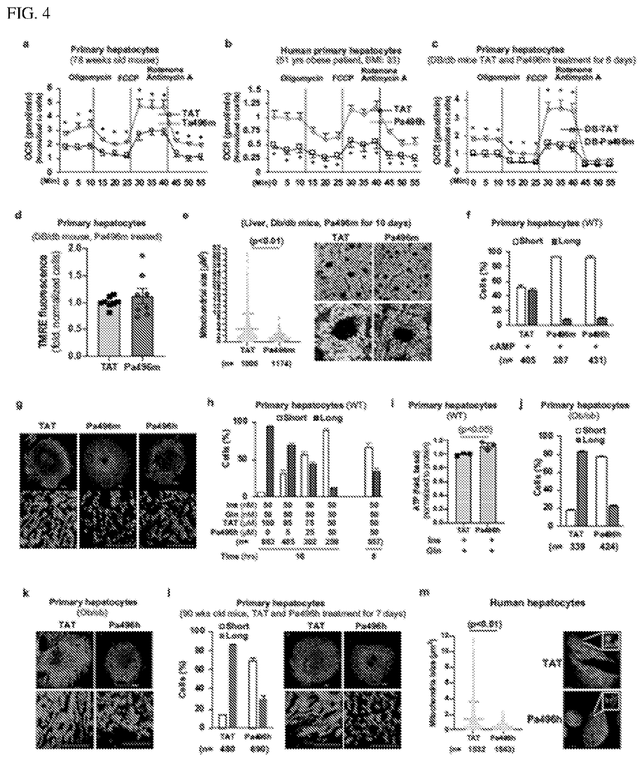

In human AMPKα1, the amino acid following S496 is valine (FIG. 2o), thus Pa496h peptide, in which the isoleucine in Pa496m was substituted with valine, was generated. Since Pa496m could activate the AMPK-MFF signaling (FIG. 3p), it was examined whether it could affect mitochondrial respiration and fission. It was found that Pa496m (5 μM) significantly increased mitochondrial respiration in primary hepatocytes prepared from elderly mouse (78 weeks of age) (FIG. 4a). Moreover, Pa496h (25 μM) treatment significantly increased mitochondrial respiration in primary hepatocytes prepared from an obese patient (FIG. 4b). In an in vivo study, db/db mice were treated with Pa496m or control TAT peptide via intraperitoneal injection (15 nMol/g/day) for 6 days, and then prepared primary hepatocytes from these mice. Primary hepatocytes prepared from Pa496m treated db/db mouse had significantly higher mitochondrial oxygen consumption rate (FIG. 4c). However, Pa496m treatment did not reduce the mitochondrial membrane potential (FIG. 4d). In another in vivo study, treatment of db/db mice with Pa496m via intraperitoneal injection (15 nMol/g/day) for 10 days significantly reduced mitochondrial size in the liver (FIG. 4e). Both Pa496m and Pa496h (50 μM) could effectively trigger the fragmentation of mitochondria in primary hepatocytes treated with 0.4 mM cAMP (FIGS. 4f, g). Pa496h triggered mitochondria fragmentation is in a dose- and time-dependent manner (FIG. 4h). Pa496h (50 μM) treatment significantly increased cellular ATP levels (FIG. 4i). In liver hepatocytes of obese ob/ob mice, the majority of the mitochondria are in an elongated form, treatment with Pa496h (100 μM) drastically increased the ratio of shorten mitochondria (FIGS. 4j, k). In an in vivo study, aged mice (90 weeks of age) treated with Pa496h through intraperitoneal injection (15 nMol/g/day) for 7 days significantly increased the ratio of shorten form of mitochondria (FIG. 4l). Pa496h (50 μM) treatment could significantly reduce mitochondrial size in human primary hepatocytes treated with 50 nM of insulin and glucagon (FIG. 4m). These data showed that treatment with either Pa496m or Pa496h augmented the ratio of the shortened form of mitochondria in hepatocytes of elderly or obese mice as well as the reduction of mitochondrial size in the liver.

### Pa496h Treatment Eliminates Comprised Mitochondria

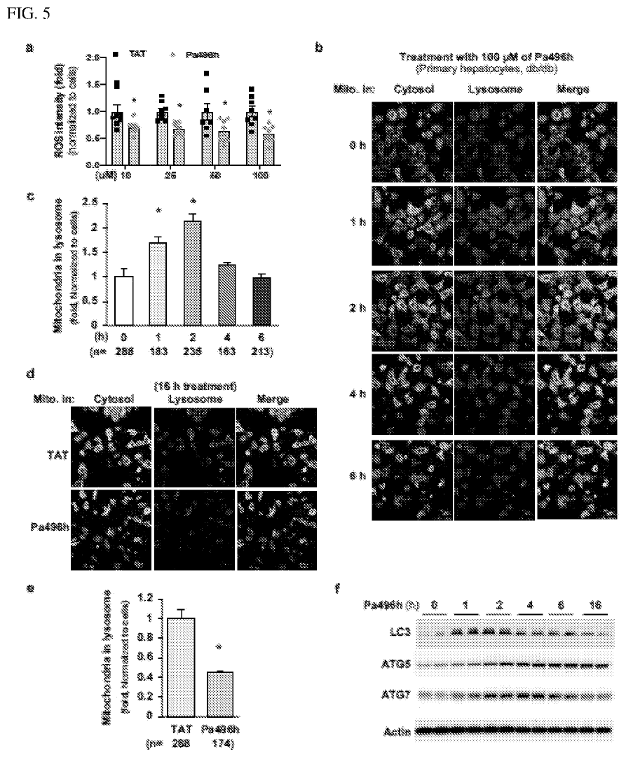

Continued and sequential cycle of mitochondrial fusion and fission is critical for maintaining a healthy mitochondrial population. It was examined whether Pa496h-stimulated mitochondrial fission could help to maintain a healthy mitochondrial population. First, after treatment with control peptide TAT and Pa496h, the primary hepatocytes prepared from elderly mouse (78 weeks of age) were stained with fluorogenic CellROX probes. Treatment with Pa496h (16 h) significantly reduced reactive oxygen species (ROS) in a concentration-dependent manner (FIG. 5a). Furthermore, using a pH dependent fluorescent protein mt-Keima ((Sun, N. et al. A fluorescence-based imaging method to measure in vitro and in vivo mitophagy using mt-Keima. Nat Protoc 12, 1576-1587, doi:10.1038/nprot.2017.060 (2017)), it was found that Pa496h (100 μM) treatment increased Keima tagged mitochondria in lysosome for degradation, that reached a peak after 2 h of treatment (FIGS. 5b, c). However, after 16 h of treatment, Keima-tagged mitochondria in lysosome were significantly reduced, indicating the elimination of comprised mitochondria (FIGS. 5d, e). In a time-course experiment, Pa496h (100 μM) activated mitophagy starting at 1 h of treatment (FIG. 5f). The above data indicate that Pa496h can increase mitophagic flux to maintain a healthy mitochondrial population.

### Pa496m and Pa496h Improve Hyperglycemia in Obese Mice

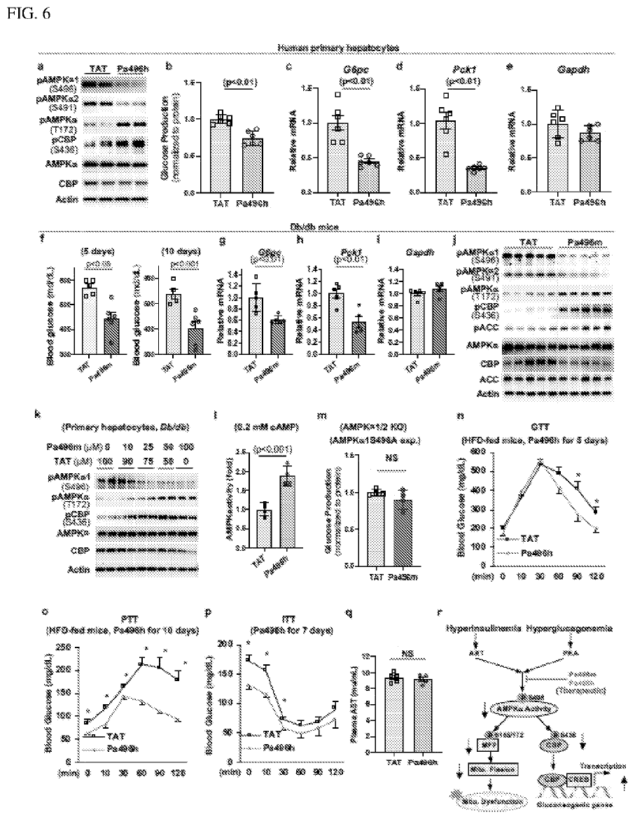

Previous studies showed that activation of AMPK can suppress liver glucose protein, leading to the improvement of hyperglycemia in obesity and T2DM ((Cao, J. et al., Low concentrations of metformin suppress glucose production in hepatocytes through AMP-activated protein kinase (AMPK). J Biol Chem 289, 20435-20446, doi:10.1074/jbc.M114.567271 (2014); Djouder, N. et al. PKA phosphorylates and inactivates AMPKalpha to promote efficient lipolysis. EMBO J 29, 469-481, doi:10.1038/emboj.2009.339 (2010)). Since the blockade of AMPKα1 phosphorylation at S496 by Pa496m resulted in the activation of AMPK in primary hepatocytes prepared from either db/db mice, cAMP or insulin/glucagon-treated hepatocytes (FIGS. 3o-q), it was tested whether Pa496h or Pa496m could suppress glucose production in primary hepatocytes. Pretreatment with 50 μM of Pa496h blocked AMPKα1/2 phosphorylation at S496/491, resulting in AMPK activation (FIG. 6a). This treatment significantly decreased glucose production (FIG. 6b) and suppressed the mRNA levels of G6pc and Pck1 in human primary hepatocytes treated with 0.2 mM cAMP (FIGS. 6c-e).

To test whether Pa496m could affect liver glucose production and improve hyperglycemia in obesity, obese db/db mice were treated with Pa496m (15 nMol/g/day) through intraperitoneal injection for 10 days. Treatment with Pa496 significantly improved hyperglycemia (FIG. 60 and suppressed the expression of rate-limiting glucogenic genes, G6pc and Pck1, in the liver (FIGS. 6g-i). Pa496m treatment blocked the phosphorylation of AMPKα1 at S496 and AMPK02 at S491, and augmented the phosphorylation of CBP at S436 in the liver (FIGS. 6j). In primary hepatocytes prepared from obese db/db mice, the blockade of AMPKα1 phosphorylation at S496 by Pa496m led to the activation of AMPK and CBP phosphorylation at S436 in a concentration-dependent manner (FIG. 6k). Treatment with 50 μM Pa469h also increased the enzymatic activity of AMPK in cAMP-treated primary hepatocytes (FIG. 6l). However, Pa496m (50 μM) treatment could not significantly suppressed cAMP-stimulated glucose production in mouse primary hepatocytes with expression of AMPKα1S496A mutant protein (FIG. 6m). In HFD-fed mice, treatment with Pa496h (10 nMole/g/day for 5 days, then 15 nMole/g/day for another 5 days) through intraperitoneal injection improved glucose tolerance, insulin sensitivity, and suppressed liver glucose production (FIGS. 6n-p) without causing injury to the liver (FIG. 6q). The above data support that activation of AMPK by Pa496m or Pa496h suppresses glucose production in hepatocytes through increasing the phosphorylation of CBP at S436, which results in the disassembly of the CREB-coactivators complex (FIG. 6r).

### Example 2

The upstream kinase LKB1 for the activation of AMPK is a tumor suppressor and is often mutated or deleted in cancers (Ross, F. A. et al. AMP-activated protein kinase: a cellular energy sensor that comes in 12 flavours. FEBS J 283, 2987-3001, doi; 10.1111/febs.13698 (2016)) and AMPK can suppress the tumorigenesis (Phoenix, K. N. et al. AMPKalpha2 Suppresses Murine Embryonic Fibroblast Transformation and Tumorigenesis. Genes Cancer 3, 51-62, doi: 10.1177/1947601912452883 (2012)). The phosphorylation of AMPKα1 at S496 and AMPK02 at S491 negatively regulates AMPK activity, three peptides can block the phosphorylation of these sites to activate AMPK were designed. AMPK is a master regulator of cellular energy metabolism (Hardie, D. G., Ross, F. A., and Hawley, S. A. AMPK: a nutrient and energy sensor that maintains energy homeostasis. Nat Rev Mol Cell Biol 13, 251-262, doi: 10.1038/nrm3311 (2012)). Activated AMPK directly phosphorylates ACC1/2 to inhibit de novo lipogenesis and mice with mutations in AMPK-targeted phosphorylation sites in ACC1/2 exhibited increased liver lipids contents (Fullerton, M. D. et al. Single phosphorylation sites in Acc1 and Acc2 regulate lipid homeostasis and the insulin sensitizing effects of metformin. Nat Med 19, 1649-1654, doi: 10.1038/nm.3372 (2013)).

(SEQ ID NO: 1, termed Pa496h)
YGRKKRRQRRRTPQRSGSVSNYRSCQR;
(SEQ ID NO: 2, termed Pa496m)
YGRKKRRQRRRTPQRSGSISNYRSCQR;
(SEQ ID NO: 3, termed Pa2-491)
YGRKKRRQRRRTPQRSCSAAGLHR;
(SEQ ID NO: 4, termed a1h)
TPQRSGSVSNYRSCQR;
(SEQ ID NO: 5, termed a1m)
TPQRSGSISNYRSCQR;
(SEQ ID NO: 6, termed a2mh)
TPQRSCSAAGLHR

A cell penetrating TAT sequence YGRKKRRQRRR (underlined) is added at the N-terminal end of some peptides to facilitate the entry into the cells, and this peptide served as control in the study.

### Blocking AMPKα1/2 Phosphorylation at S496/491 Inhibits the Growth of Tumor Cells

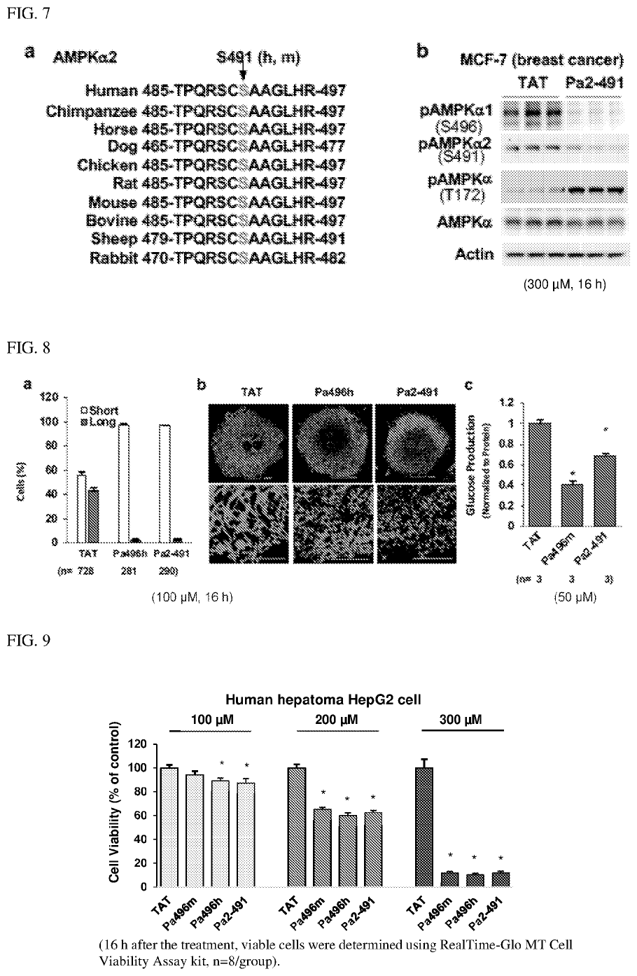

As shown in Example 1, both Pa496m and Pa496h are able to activate AMPK. The serine 491 phosphorylation site in AMPKα2, corresponding phosphorylation of AMPKα1 at S496, is conserved across eukaryotic species (FIG. 7a). It was found that Pa2-491 (300 μM, 16) could block the phosphorylation of AMPKα1S496 and AMPKα2S491 and activate AMPK by increasing the phosphorylation of AMPKα at T172 (biomarker of AMPK activation) in breast cancer MCF-7 cells (FIG. 7b).

Both Pa496m and Pa496h robustly trigger mitochondrial fission (FIGS. 4e-m) and Pa2-491 can trigger mitochondrial fission to a similar degree as Pa496h (FIG. 8a, b). Furthermore, Pa2-491 could significantly suppress glucose production in primary hepatocytes prepared from elderly mice (78 weeks of age), however, to less degree compared to Pa496m (FIG. 8c).

To test whether blockade of AMPKα 1/2 phosphorylation at S496/491 had any effects on the growth of tumor cells, human hepatoma HepG2 cells were treated with control TAT, Pa496m, Pa496h, and Pa2-491 at 100 μM or 200 μm for 16 h. It was found that treatment with 100 μM of Pa496h and Pa2-491 significantly decreased the tumor cell growth by 10.7% and 12.5% respectively (FIG. 9). Treatment with 200 μM of Pa496m, Pa496h, and Pa2-491 had a greater inhibition of the tumor cell growth, and decreased the tumor cell growth by 34.5%, 40%, and 37.2%, respectively. Treatment with 300 μM of Pa496m, Pa496h, and Pa2-491 suppressed near 90% of tumor cell growth.

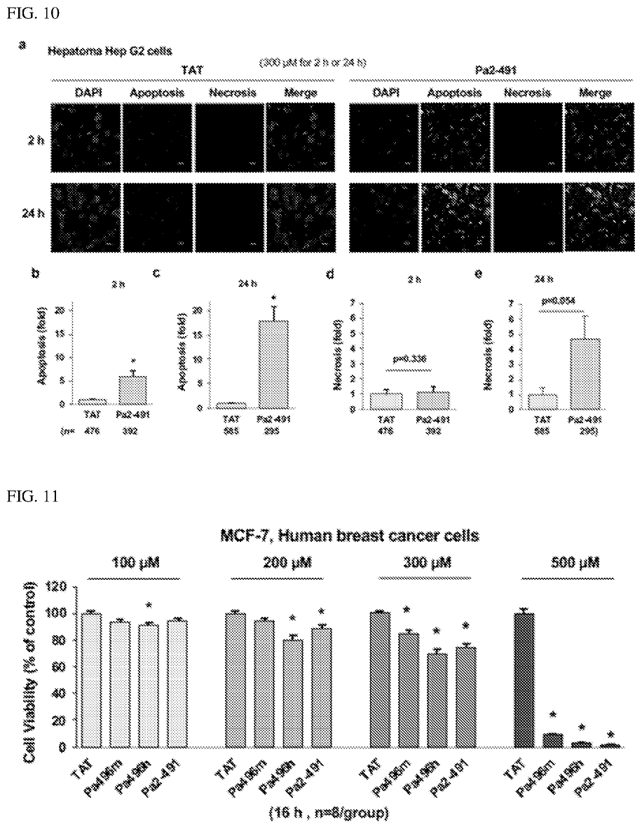

Having seen that treatment of Pa496m, Pa496h, and Pa2-491 decreased the viable tumor cells (FIG. 9), it was examined whether the inhibition is through increasing the apoptosis and/or necrosis. Hepatoma HepG2 cells were treated 300 μM of Pa2-491 for 2 h or 24 h, it was found that Pa2-491 treatment increased the apoptosis by over 16-fold (24 h treatment) compared to the treatment with control TAT treatment (FIGS. 10a-c). Increased necrosis was observed in HepG2 cells treated with Pa2-491 however, the level did not reach the statistical significance (FIGS. 10 d, e).

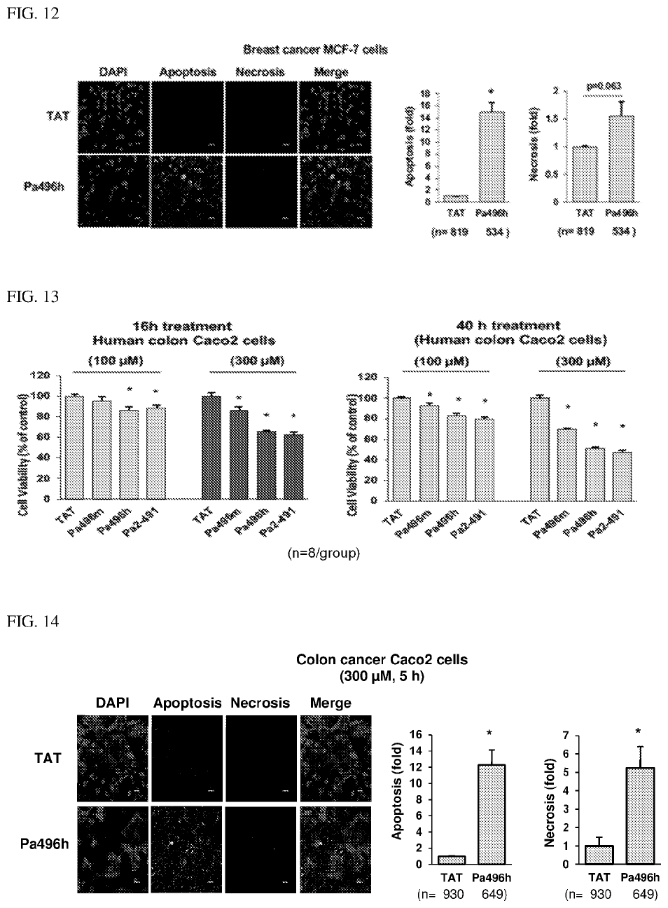

It was found that Pa496m, Pa496h, and Pa2-491 could decrease the number of viable MCF-7 breast cancer cells (FIG. 11). Pa496h has the strongest effects of inhibition at lower concentrations (100, 200, 300 μM), indicating that individual kind of cancer cells may have a different response to these agents. At a concentration of 500 μM, treatment with each of Pa496m, Pa496h, and Pa2-491 led to more than 90% of reduction of vial MCF-7 cancer cells. It was found that Peptide Pa496h (300 μM, 4 h) could activate apoptosis, but not the necrosis in breast cancer MCF-7 cells (FIG. 12).

It was tested whether Pa496m, Pa496h, and Pa2-491 could inhibit the growth of human colon cancer Caco2 cells (FIG. 13). Treatment with Pa496h and Pa2-491 significantly reduced the viable cancer cell numbers at 100 μM. However, at 300 μM concentration, all three agents significantly decreased the viable cancer cell numbers. Of note, the inhibition of tumor cell growth is also time-dependent because treatment for 40 h had a stronger inhibition (Compare right panel to left panel in FIG. 13). Peptide Pa496h could activate apoptosis in colon cancer Caco2 tumor cells (FIG. 14).

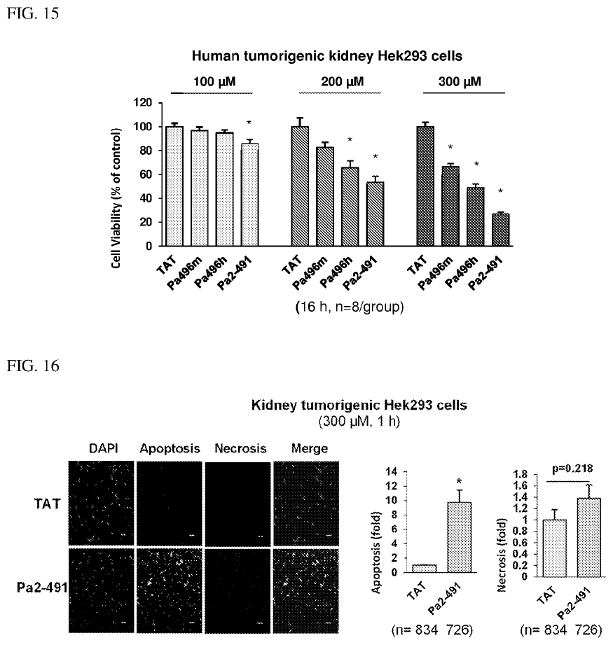

In human tumorigenic kidney Hek293 cells, treatment with 100 μM of Pa2-491 significantly decreased the viable cell number of Hek293 cells, at 200 μM concentration, both Pa496h and Pa2-491 significantly decreased the viable cell number of Hek293 cells (FIG. 15). Peptide Pa2-491 could activate apoptosis in Hek293 cells (FIG. 16).

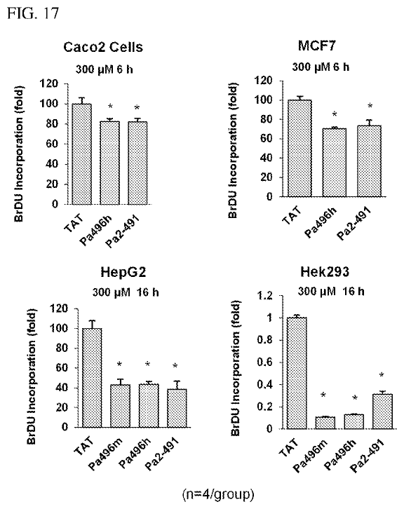

To further confirm that these peptides are able to inhibit the growth of cancer cells, a BrDu incorporation assay was performed (FIG. 17). Treatment with 300 μM of Pa496m, Pa496h, or Pa2-491 for either 6 h or 16 h significantly decreased tumor cells' proliferation.

### Blocking AMPKα1/2 Phosphorylation at S496/S491 Alleviates the Fat Accumulation in Hepatocytes

Excessive accumulation of triglycerides (TG) is a hallmark of nonalcoholic fatty liver disease (NAFLD). NAFLD is rising rapidly and is the leading indication for liver transplantation worldwide. NAFLD affects over 30% of the population in the USA ((Younossi, Z. M. et al. Global epidemiology of nonalcoholic fatty liver disease-Meta-analytic assessment of prevalence, incidence, and outcomes. Hepatology 64, 73-84, doi: 10.1002/hep.28431 (2016)). NAFLD is now recognized as the liver disease component of metabolic syndrome because patients with NAFLD often have obesity and type 2 diabetes mellitus (T2DM) with insulin resistance, dyslipidemia, hypertriglyceridemia, and hypertension ((Li, Z. et al. Prevalence of nonalcoholic fatty liver disease in mainland of China: a meta-analysis of published studies. J Gastroenterol Hepatol 29, 42-51, doi: 10.1111/jgh.12428 (2014)). In patients with T2DM, over 75% of patients have a NAFLD, and over 90% of severely obese patients underwent bariatric surgery have NAFLD ((Portillo Sanchez, P. et al. High Prevalence of Nonalcoholic Fatty Liver Disease in Patients with Type 2 Diabetes Mellitus and Normal Plasma Aminotransferase Levels. J Clin Endocrinol Metab 100, doi: 10.1210/jc.2014-2739 (2014); Kasturiratne, A. et al. Influence of non-alcoholic fatty liver disease on the development of diabetes mellitus. J Gastroenterol Hepatol 28, 142-147, doi: 10.1111/j.1440-1746.2012.07264.x (2013)).

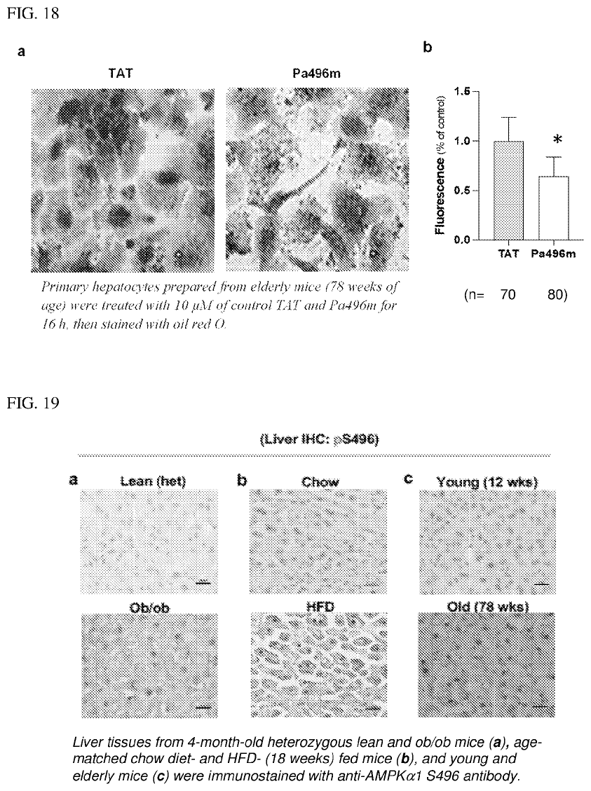

It was examined whether the activation of AMPK by blocking AMPKα1/2 phosphorylation at S496/491 could decrease the fat accumulation in hepatocytes. Primary hepatocytes prepared from elderly mice (78 weeks of age) were treated with 10 μM of control peptide TAT or Pa496m for 16 h. Treatment with Pa496m significantly decreased fat content in hepatocytes (FIG. 18).

### Example 3

In Example 1, data showing that elevated blood levels of insulin and glucagon stimulate the phosphorylation of AMPKα1 at S496 and/or AMPKα2 at S491, subsequently leading to the impairment of AMPK activity and mitochondrial fission is provided. To expand upon these findings, the phosphorylation status of AMPKα1 at S496 was determined in liver tissue because this site is the peptide-targeting site. It was found that immunostained liver sections from obese ob/ob mice, high-fat-diet (HFD)-induced obese mice, and elderly mice had increased AMPKα1 phosphorylation at S496 (FIG. 19).

Given that these peptides could significantly suppress the growth of breast cancer' and liver cancer' cells (FIGS. 9, 11), the phosphorylation status of AMPKα1 at S496 in human breast cancer tissue and liver cancer tissue was determined. A drastically increased phosphorylation of AMPKα1 at S496 in both human liver cancer tissues (FIG. 20a) and breast cancer tissues (FIG. 20b) was identified.

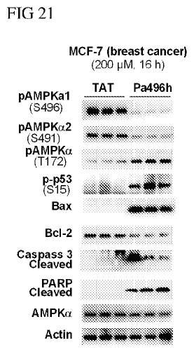

Activated AMPK can phosphorylate p53 at S15 and trigger the apoptosis by p53 (Nieminen, A. et al. Myc-induced AMPK-phospho p53pathway activates Bak to sensitize mitochondrial apoptosis. PNAS 110, E1839-1848, doi: 10.1073/pnas. 1208530110 (2013)). It was examined whether activation of AMPK by Pa496h could affect p53 phosphorylation at S15, thus resulting in the apoptosis. It was found that treatment with Pa496h increased the phosphorylation of p53 at S15 and activated the signaling pathway of apoptosis in MCF7 cells (FIG. 21).

### Example 4

### AMPK-Targeting Peptides Inhibit the Growth of Human Primary Dissociated Tumor Cells (DTCs)

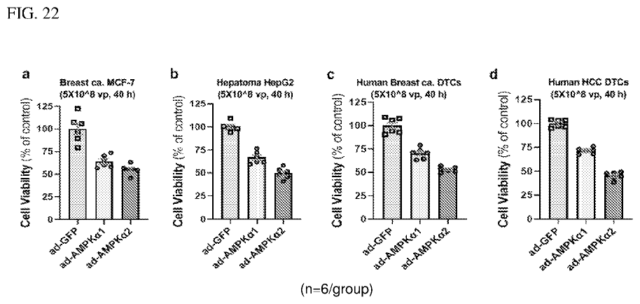

Patient's tumors with decreased AMPK activity correlate with poor prognosis and hepatocellular carcinoma cells with knockdown of AMPK had greater tumorigenicity when implanted into nude mice ((Zheng, L. et al. Prognostic significance of AMPK activation and therapeutic effects of metformin in hepatocellular carcinoma. Clin Cancer Res 19, 5372-5380, doi:10.1158/1078-0432.CCR-13-0203 (2013); Kim, H. S. et al. Berberine-induced AMPK activation inhibits the metastatic potential of melanoma cells via reduction of ERK activity and COX-2 protein expression. Biochem Pharmacol 83, 385-394, doi:10.1016/j.bcp.2011.11.008 (2012)). It was tested whether AMPK could suppress tumor cells' growth, and was found that overexpressing of either AMPK catalytic α1 or α2 subunit significantly decreased the growth of breast cancer MCF-7 cells (FIG. 22a), hepatoma HepG2 cells (FIG. 22b), human primary breast cancer (invasive ductal carcinoma) dissociated tumor cells (Breast-cancer-DTCs) (FIG. 22c), and human primary hepatocellular carcinoma dissociated tumor cells (HCC-DTCs) (FIG. 22d).

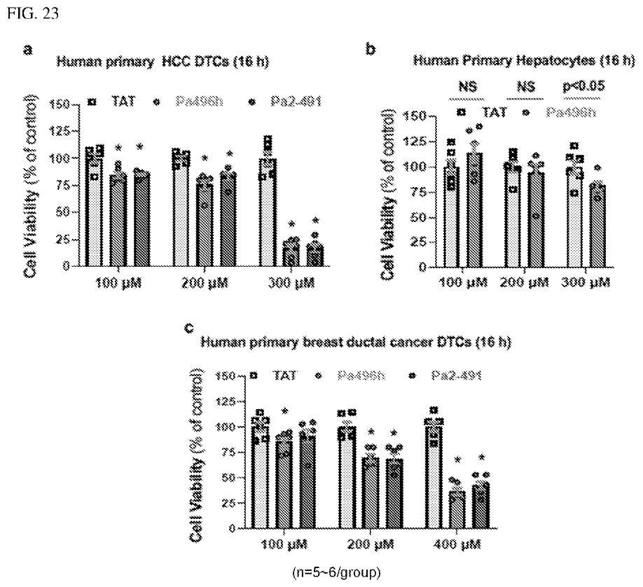

To test whether blockade of AMPKα1/2 phosphorylation at S496/491 had any effects on the growth of human primary HCC-DTCs, human primary HCC-DTCs were treated with control TAT, Pa496h, and Pa2-491 at 100 μM, 200 μM, or 300 μM for 16 h. It was found that treatment with 100 μM of Pa496h and Pa2-491 significantly decreased the tumor cell growth by 15.4% and 16.2% respectively (FIG. 23a). Treatment with 200 μM of Pa496h and Pa2-491 decreased the tumor cell growth by 24.5%, and 17.2%, respectively. Treatment with 300 μM of Pa496h and Pa2-491 had a more pronounced inhibition of the tumor cell growth, and decreased the tumor cell growth by 83.2%, and 83.1%, respectively.

However, human primary hepatocytes could tolerate the treatment of Pa496h. It was found that treatment with either 100 μM or 200 mM of Pa496h did not significantly affected the viability of human primary hepatocytes (FIG. 23b), only treatment with 300 PM of Pa496h decreased hepatocytes' viability by 19.8%, indicating that primary hepatocytes were well tolerated the treatment of Pa496h (Compare FIG. 22a to FIG. 22b).

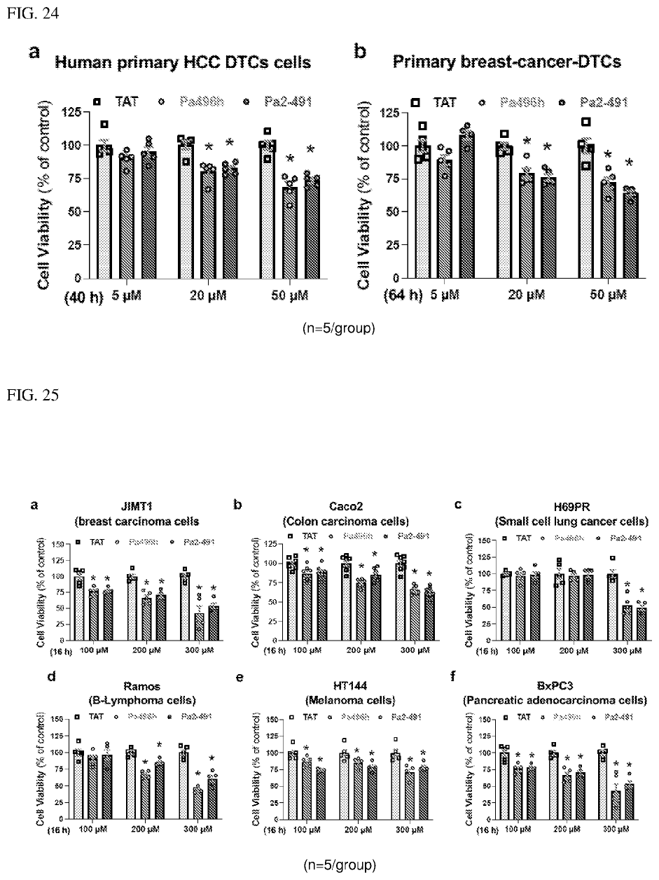

Furthermore, human primary breast-cancer-DTCs were treated with control TAT, Pa496h, and Pa2-491 at 100 μM, 200 μM, or 400 μM for 16 h. It was found that treatment with 100 μM of Pa496h and Pa2-491 decreased the growth of breast cancer-DTCs by 14.7% and 9.3% respectively (FIG. 23c). Treatment with 200 μM of Pa496h and Pa2-491 had a greater inhibition of the tumor cell growth, and significantly decreased the tumor cell growth by 30.4%, and 31.3%, respectively. Treatment with 400 μM of Pa496h and Pa2-491 had a more pronounced inhibition of the tumor cell growth, and decreased the tumor cell growth by 63.5%, and 57.8%, respectively. Treatment with lower concentration (20 μm) of Pa496h or Pa2-491 for 40 h also significantly reduced the growth of human primary HCC-DTCs (FIG. 24a). Treatment with lower concentration (20 μm) of Pa496h or Pa2-491 for 64 h also significantly reduced the growth of human primary breast-cancer-DTCs (FIG. 24b).

It was found that Pa496h and Pa2-491 could inhibit the growth of another breast cancer JIMT1 cells (FIG. 25a), colon carcinoma Caco2 cells (FIG. 25b), small cell lung cancer H69PR cells (FIG. 25c), B lymphoma cells (FIG. 25d), melanoma HT144 cells (FIG. 25e), and pancreatic adenocarcinoma BxPC3 cells (FIG. 25f).

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

We claim:

1. A method of blocking the phosphorylation of AMPKalpha1 at serine 496 and/or AMPKalpha2 at serine 491, comprising:
contacting said AMPKalpha with a peptide that specifically blocks phosphorylation of said AMPKalpha 1 at serine 496 and/or AMPKalpha2 at serine 491.

2. The method of claim 1, wherein said AMPKalpha is in a cell.

3. The method of claim 2, wherein said cell is in vivo or in vitro.

4. The method of claim 2 or 3, wherein said contacting increases mitochondrial oxidative activity in said cell.

5. The method of claim 3 or 4, wherein said contacting treats or prevents a metabolic disorder.

6. The method of claim 5, wherein said metabolic disorder is selected from the group consisting of diabetes, obesity, cardiovascular diseases, and nonalcoholic fatty liver disease (NAFLD).

7. The method of claim 3 or 4, wherein said contacting treats cancer.

8. A method of improving one or more measures of mitochondrial function in a subject, comprising:
administering a peptide that specifically blocks phosphorylation of AMPKalpha1 at serine 496 and/or AMPKalpha2 at serine 491 to said subject.

9. The method of claim 8, wherein said measures of mitochondrial function are selected from the group consisting of increasing mitochondrial respiration, enhancing mitochondrial fission, and improving mitochondrial oxidative state.

10. A method of treating or preventing a metabolic disorder in a subject, comprising:
administering a peptide that specifically blocks phosphorylation of AMPKalpha1 at serine 496 and/or AMPKalpha2 at serine 491 to said subject.

11. The method of claim 10, wherein said metabolic disorder is selected from the group consisting of diabetes, obesity, cardiovascular diseases, and NAFLD.

12. A method of treating cancer in a subject, comprising:
administering a peptide that specifically blocks phosphorylation of AMPKalpha1 at serine 496 and/or AMPKalpha2 at serine 491 to said subject.

13. The method of any of the preceding claims, wherein said peptide is selected from the group consisting of YGRKKRRQRRRTPQRSGSVSNYRSCQR (SEQ ID NO:1); YGRKKRRQRRRTPQRSGSISNYRSCQR (SEQ ID NO:2); YGRKKRRQRRRTPQRSCSAAGLHR (SEQ ID NO:3); TPQRSGSVSNYRSCQR (SEQ ID NO:4); TPQRSGSISNYRSCQR (SEQ ID NO:5); TPQRSCSAAGLHR (SEQ ID NO:6) and peptides with at least 90% identity to SEQ ID NOs: 1-6.

14. The method of claim 13, wherein said peptide is at least 95% identical to SEQ ID NOs: 1-6.

15. The method of claim 13, wherein said peptide is at least 95% identical to SEQ ID NOs: 1-6.

16. The method of claim 13, wherein said peptide comprises, consisting essentially of, or consists of a peptide selected from the group consisting of SEQ ID NOs: 1-6.

17. The use of a peptide that specifically blocks phosphorylation of AMPKalpha 1 at serine 496 and/or AMPKalpha2 at serine 491 to improve one or more measures of mitochondrial function, treat or prevent a metabolic disorder, or treat cancer in a subject.

18. A composition, comprising
A peptide selected from the group consisting of YGRKKRRQRRRTPQRSGSVSNYRSCQR (SEQ ID NO:1); YGRKKRRQRRRTPQRSGSISNYRSCQR (SEQ ID NO:2); YGRKKRRQRRRTPQRSCSAAGLHR (SEQ ID NO:3); TPQRSGSVSNYRSCQR (SEQ ID NO:4); TPQRSGSISNYRSCQR (SEQ ID NO:5); TPQRSCSAAGLHR (SEQ ID NO:6) and peptides with at least 90% identity to SEQ ID NOs: 1-6.

19. The composition of claim 18, wherein said composition is a pharmaceutical composition.


SEQUENCE LISTING (7 sequences)

SEQ ID NO: 1 - protein, 27 aa, synthetic construct
YGRKKRRQRRRTPQRSGSVSNYRSCQR

SEQ ID NO: 2 - protein, 27 aa, synthetic construct
YGRKKRRQRRRTPQRSGSISNYRSCQR

SEQ ID NO: 3 - protein, 24 aa, synthetic construct
YGRKKRRQRRRTPQRSCSAAGLHR

SEQ ID NO: 4 - protein, 16 aa, synthetic construct
TPQRSGSVSNYRSCQR

SEQ ID NO: 5 - protein, 16 aa, synthetic construct
TPQRSGSISNYRSCQR

SEQ ID NO: 6 - protein, 13 aa, synthetic construct
TPQRSCSAAGLHR

SEQ ID NO: 7 - protein, 11 aa, synthetic construct
YGRKKRRQRRR